(19) United States
(12) Patent Application Publication
(10) Pub. No.: US 2024/0207094 A1
(43) Pub. Date: Jun. 27, 2024

(54) IRRIGATION DAMPING IN PHACOEMULSIFICATION

(71) Applicant: JOHNSON & JOHNSON SURGICAL VISION, INC., Irvine, CA (US)

(72) Inventors: Assaf Govari, Haifa (IL); Vadim Gliner, Haifa (IL); Ilya Sitnitsky, Nahariya (IL); Andres Claudio Altmann, Haifa (IL); Alexander Shechtman, Haifa (IL); Elad Avraham Diukman, Haifa (IL); Alon Boumendil, Givat Nili (IL); Sergey Sobol, Haifa (IL)

(21) Appl. No.: 18/089,412

(22) Filed: Dec. 27, 2022

(51) Int. Cl. A61F 9/007

(52) U.S. Cl. CPC A61F 9/00745; A61B 2217/005; A61B 2217/007; A61M 25/1029

(57) ABSTRACT

A phacoemulsification system includes a phacoemulsification probe, an aspiration tube and an irrigation tube. The aspiration tube is connectable to the phacoemulsification probe to aspirate a cataract from an eye. The irrigation tube is connectable to the probe and, in one embodiment, has at least one integrated compliant tube section at an end near the phacoemulsification probe. Each integrated compliant tube section is less than 1% of the length of the irrigation tube, has a section diameter larger than a tube diameter of the irrigation tube, and is more compliant than a cornea of the eye. In another embodiment, a compliant tube section is connectable to an end of the irrigation tube and to the probe and is formed of a material more compliant than the irrigation tube.

### FIELD OF THE INVENTION

The present invention relates to phacoemulsification systems generally and to irrigation damping in them in particular.

### BACKGROUND OF THE INVENTION

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery.

In the procedure, the surgeon makes a small incision in the sclera or cornea of the eye. Then a portion of the anterior surface of the lens capsule is removed to gain access to the cataract. The surgeon then uses a phacoemulsification probe, which is an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution (BSS) to maintain the anterior chamber of the eye. After removing the cataract with phacoemulsification, the softer outer lens cortex is removed with suction. An intraocular lens (IOL) is then introduced into the empty lens capsule restoring the patient's vision.

### SUMMARY OF THE PRESENT INVENTION

There is provided, in accordance with a preferred embodiment of the present invention, a phacoemulsification system including a phacoemulsification probe, an aspiration tube and an irrigation tube. The aspiration tube is connectable to the phacoemulsification probe to aspirate a cataract from a cornea. The irrigation tube is connectable to the phacoemulsification probe and has at least one integrated compliant tube section at an end near the phacoemulsification probe. Each integrated compliant tube section is less than 1% of a length of the irrigation tube, has a section diameter larger than a tube diameter of the irrigation tube, and is more compliant than the cornea.

There is also provided, in accordance with a preferred embodiment of the present invention, an irrigation tube for a phacoemulsification probe. The irrigation tube includes a long tube portion, and at least one integrated compliant tube section at an end of the long tube portion near its connection to the phacoemulsification probe. Each integrated compliant tube section is less than 1% of a length of the irrigation tube, has a section diameter larger than a tube diameter of the irrigation tube, and is more compliant than the cornea.

Moreover, in accordance with a preferred embodiment of the present invention, the at least one integrated compliant tube section is at least one balloon integrally formed in the irrigation tube.

Further, in accordance with a preferred embodiment of the present invention, the at least one balloon is produced by blow molding of the irrigation tube.

Still further, in accordance with a preferred embodiment of the present invention, the section diameter is 10-50% larger than the tube diameter.

There is also provided, in accordance with a preferred embodiment of the present invention, a phacoemulsification system having a phacoemulsification probe. The system includes an aspiration tube, an irrigation tube and a compliant tube section. The aspiration tube is connectable to the phacoemulsification probe to aspirate a cataract from a cornea. The compliant tube section is connectable to an end of the irrigation tube and to the phacoemulsification probe. The compliant tube section has a section diameter larger than a tube diameter of the irrigation tube, and is more compliant than the cornea.

There is also provided, in accordance with a preferred embodiment of the present invention, an irrigation tube for a phacoemulsification probe. The irrigation tube includes a long tube portion, and a compliant tube section connectable to an end of the long tube portion and to the phacoemulsification probe. The compliant tube section has a section diameter larger than a tube diameter of the irrigation tube, and is more compliant than the cornea.

Moreover, in accordance with a preferred embodiment of the present invention, the compliant tube section is formed of silicone with a Shore value of 70-80.

Further, in accordance with a preferred embodiment of the present invention, the compliant tube section has a length of 3-10 cm long.

Finally, in accordance with a preferred embodiment of the present invention, the compliant tube section is formed of a material more compliant than a material of the irrigation tube.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a phacoemulsification system:

FIG. 2 is a schematic illustration of a phacoemulsification system having a wide tube section of an irrigation tube, constructed and operative in accordance with a preferred embodiment of the present invention:

FIGS. 3A and 3B are schematic illustrations of a single and multiple integrated balloon tube section(s), respectively, forming the wide tube section of FIG. 2:

FIG. 3C is a schematic cross-section of the integrated balloon tube section of FIG. 3A:

FIG. 4 is a schematic illustration of a compliant tube section forming the wide tube section of FIG. 2; and

FIG. 5 which is a graphical illustration of the intraocular pressure using the wide tube section of FIG. 2.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

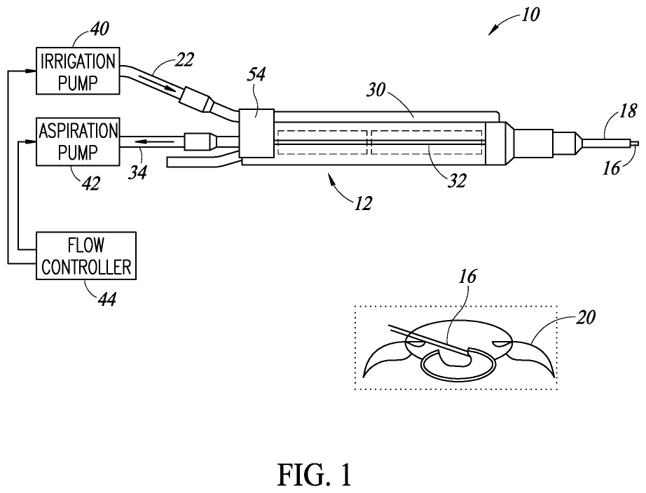

Reference is now made to FIG. 1, which illustrates a phacoemulsification system 10 comprising a phacoemulsification probe 12 which comprises a needle 16, a probe body 17, and an irrigation sleeve 18 that surrounds needle 16 and creates a fluid pathway between the external wall of needle 16 and the internal wall of irrigation sleeve 18. Needle 16 is hollow to provide an aspiration channel, connected to an aspiration pathway 32. Moreover, irrigation sleeve 18 may have one or more side ports at, or near, its distal end to allow irrigation fluid to flow out of the ports and into the surgical site.

During the phacoemulsification procedure, an irrigation pump 40 pumps irrigation fluid from an irrigation reservoir, through an irrigation tube 22 and through an irrigation channel 30 of probe 12 to the irrigation sleeve 18 to irrigate the eye 20.

An aspiration pump 42 may aspirate eye fluid and waste matter (e.g., emulsified parts of the cataract) via aspiration pathway 32, which extends from the hollow of needle 16 through the phacoemulsification probe 12, and then via an aspiration tubing line 34 to a collection receptacle.

In some example embodiments, phacoemulsification system 10 additionally includes an Anti-Vacuum Surge (AVS) device 54 that is configured to respond to vacuum surges. Vacuum surges may occur in response to needle 16 being occluded for a given period of time due to aspiration of a relatively large particle. The occlusion leads to a vacuum buildup at a proximal in end of the occlusion. Once the occlusion is cleared, the vacuum buildup has the potential of suctioning material from the surgical site and causing damage. Anti-Vacuum Surge (AVS) device 54 is configured to cut-off the aspiration flow felt at distal end of needle 16 and releasing any vacuum buildup in response to detecting a vacuum surge.

A flow controller 44 controls the irrigation and aspiration flows separately to maintain a steady IOP (intraocular pressure). However, due to the small volume of the surgical site, even small fluctuations of either irrigation flow or aspiration flow can cause the IOP to fluctuate. Such fluctuations may be caused by vibrations introduced by the mechanical action of pumps 40 and/or 42, by a small mismatch in the flow from pumps 40 and 42 and/or by a slow response of pumps 40 and 42 to changes in actuation.

In general, during irrigation only, there are relatively few fluctuations in IOP, while, when there are both irrigation and aspiration flows, there are many, significant fluctuations.

These fluctuations may be distracting to the physician. For example, the eye chamber may noticeably expand and contract and/or fluid may periodically squirt out through the incision, both due to fluctuations in the IOP. The fluctuations are expected to be more pronounced at higher IOP levels because at high IOP levels the eye chamber is less compliant and therefore cannot act as a damper. Typically, these fluctuations have a stable frequency related to pump motion. For example, pumps 40 and 42, which may be progressive cavity pump (PCP) pumps, may have two superimposed movements—rotation and translation. Both types of motion may cause fluctuations in the IOP.

Applicant has realized that, by damping the irrigation flow, the fluctuations may be significantly reduced.

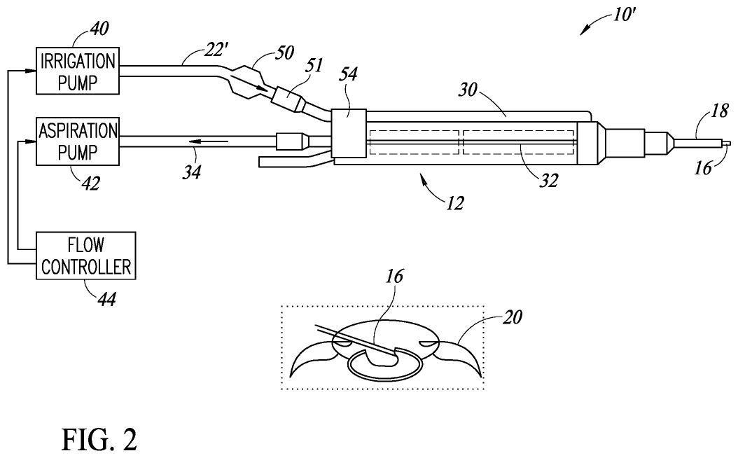

Reference is now made to FIG. 2, which illustrates a phacoemulsification system 10′, similar to phacoemulsification system 10′ but additionally comprising a wide tube section 50 in a short portion of the irrigation tube, here labeled 22″, just before or near its connection 51 to probe 12, may dampen the irrigation flow. Since wide tube section 50 is wider, as indicated by the arrows, than the much longer irrigation tube 22′, it may provide the required damping effect over its relatively short distance. In general, wide tube section 50 may be less than 10% or less than 1% of the length of irrigation tube 22″.

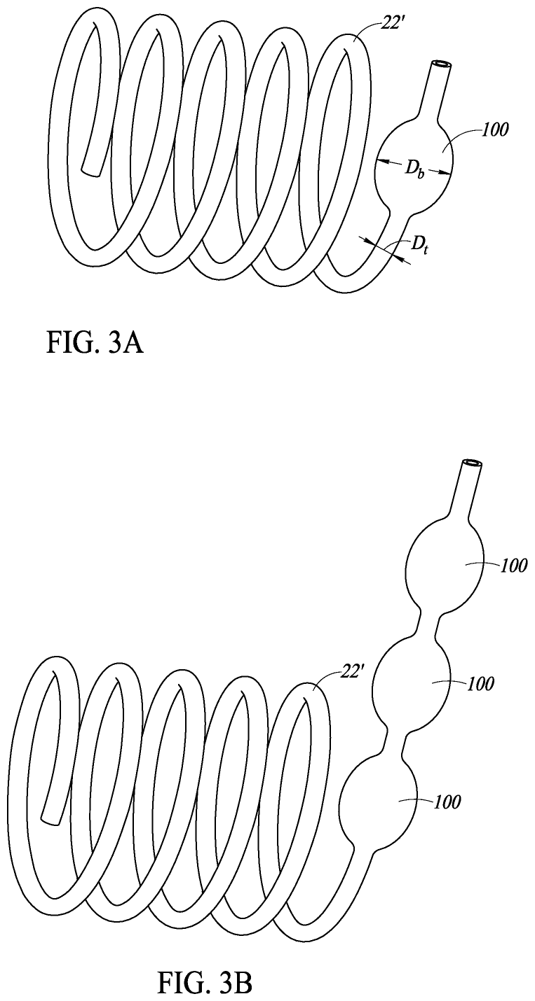

In one embodiment, shown in FIGS. 3A and 3B to which reference is now made, wide tube section 50 is an integrated balloon tube section 100 (FIG. 3A) or multiple integrated balloon tube sections 100 (FIG. 3B), formed at the distal end of irrigation tube 22′, close to connection 51. Each balloon tube section 100 may have a balloon diameter Db, which may define the amount of damping it provides, where N balloon tube sections 100 may provide N times the amount of damping of a single balloon tube section 100. Balloon diameter Do may be 2-5 times larger than a tube diameter Dt of irrigation tube 22′. For example, balloon diameter Db might be 8-30 mm while tube diameter Dt might be 4-6 mm. In this embodiment, balloon 100 might be 25-40 mm long.

Integrated balloon tube section(s) 100 may be formed from irrigation tube 22′ using a process such as blow molding, typically used to form balloon catheters, stents and ENT sinus procedures, among others. It will be appreciated that, as a result, integrated balloon tube sections 100 may be relatively inexpensive to manufacture and to maintain its sterilization.

Moreover, because of the blow molding, integrated balloon tube section(s) 100 may have thinner walls than those of irrigation tube 22. This is shown in FIG. 3C, a cross-section of one balloon section 100. Note that the walls of irrigation tube 22′ become thinner when stretched to become integrated balloon tube section(s) 100. In the example hereinabove, the thickness of walls of irrigation tube 22′ might be 0.7-1.0 mm while the thickness of the walls of integrated balloon tube section(s) 100, at their thinnest, might be 0.35-0.5 mm.

It will be appreciated that, with thinner walls, integrated balloon tube section 100 may be more compliant which may help it absorb fluctuations and/or provide damping in addition to its wider balloon diameter Db (as compared to tube diameter Dt).

The level of compliance may be designed to be greater than the compliance of the cornea. As a result, integrated balloon tube section(s) 100 will collapse in the presence of a vacuum surge instead of the cornea.

Integrated balloon tube section(s) 100 may be formed in irrigation tube 22′, where the size and number of balloons may be selected by the manufacturer based on the desired flow damping characteristics. Irrigation tube 22′ with balloon tube sections 120 may be provided with a particular probe or a surgeon may select which damped irrigation tube 22's/he may desire as a function of how much damping is necessary, given the characteristics of the phacoemulsification system and/or the characteristics of the eye to be incised. The result may be a very versatile and cost effective damping irrigation tube 22″.

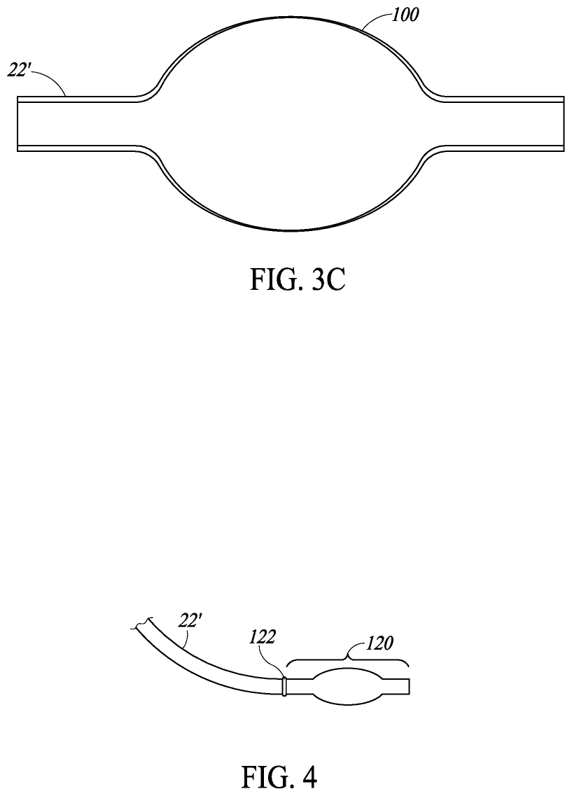

In an alternative embodiment, shown in FIG. 4 to which reference is now made, wide tube section 50 may be a separate tube section 120 (or stand-alone tube section) that is connectable to irrigation tube 22′ at one end with a connector 122 and at an opposite end to probe 12 and/or AVS 54. In other example embodiments, separate tube section 120 includes an additional connector 122 on an opposite end so that separate tube section 120 may be connected anywhere along the length of the irrigation tubing. Separate tube section 120 may be formed from the same material as irrigation tubing 22′ or from a different material. Optionally, separate tube section 120 may be formed from a material that is more compliant than irrigation tube 22′. Its higher compliance together with its wider diameter may provide an enhanced damping effect.

Since compliant tube section 120 is connectable to irrigation tube 22′, it may be formed of a very compliant material. As in the previous embodiment, this may ensure that compliant tube section 120 will collapse, in the presence of a vacuum surge, rather than the cornea. For example, compliant tube section 120 may be formed from silicone with a Shore value of 70-80 and may be between less than 1% of the length of irrigation tube 22′. For example, it might be 3-10 cm long, preferably be 5 cm long.

It will be appreciated that the compliancy may enable the compliant tube section 120 to absorb fluctuations and thereby provide damping to the fluctuations.

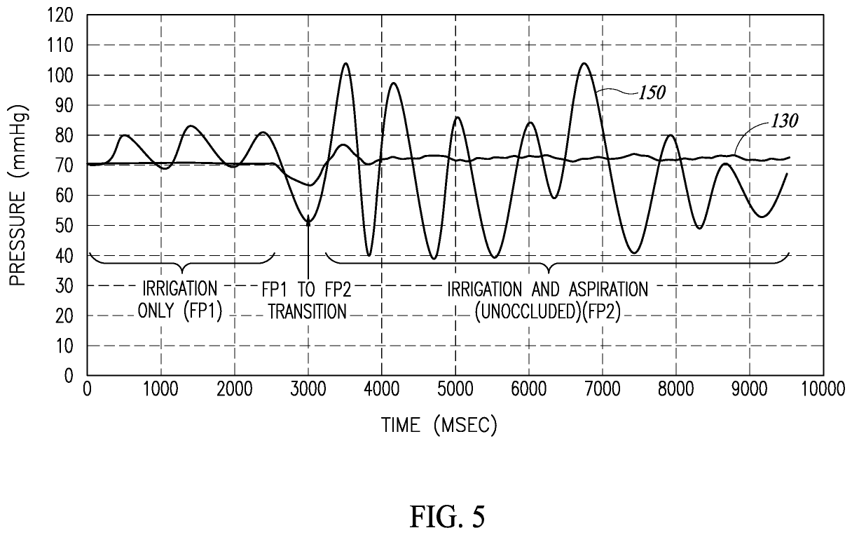

Reference is now briefly made to FIG. 5, which schematically graphs the IOP using wide tube section 50 in a graph labeled 130, and the IOP without wide tube section 50, in a graph labeled 150. As can be seen, the fluctuations of graph 150 are significantly dampened in graph 130, to the point that graph 130 is relatively flat. The only significant fluctuations are during a transition period, at about 3000 msec, between irrigation only and aspiration and irrigation together.

It will be appreciated that both integrated balloon tube section(s) 100 and compliant tube 120 may implement wide tube section 50 and that wide tube section 50 may be only a small section of different tubing. The result is an inexpensive and versatile addition to irrigation tube 22′, enabling the rest of irrigation tube 22′ to maintain the necessary attributes yet providing sufficient irrigation damping.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A phacoemulsification system comprising:
a phacoemulsification probe;
an aspiration tube connectable to said phacoemulsification probe to aspirate a cataract from an eye; and
an irrigation tube connectable to said phacoemulsification probe and having at least one integrated compliant tube section at an end near said phacoemulsification probe, wherein each said at least one integrated compliant tube section is less than 1% of a length of said irrigation tube, has a section diameter larger than a tube diameter of said irrigation tube, and is more compliant than a cornea of the eye.

2. The system according to claim 1 and wherein said at least one integrated compliant tube section is at least one balloon integrally formed in said irrigation tube.

3. The system according to claim 2 and wherein said at least one balloon is produced by blow molding of said irrigation tube.

4. The system according to claim 2 and wherein said section diameter is 10-50% larger than said tube diameter.

5. An irrigation tube for a phacoemulsification probe used for aspirating a cataract from an eye, the irrigation tube comprising:
a long tube portion; and
at least one integrated compliant tube section at an end of said long tube portion near its connection to said phacoemulsification probe,
wherein each said at least one integrated compliant tube section is less than 1% of a length of said irrigation tube, has a section diameter larger than a tube diameter of said irrigation tube, and is more compliant than a cornea of the eye.

6. The tube according to claim 5 and wherein said at least one integrated compliant tube section is at least one balloon integrally formed in said irrigation tube.

7. The tube according to claim 6 and wherein said at least one balloon is produced by blow molding of said long tube portion.

8. The system according to claim 6 and wherein said section diameter is 10-50% larger than said tube diameter.

9. A phacoemulsification system having a phacoemulsification probe, the system comprising:
an aspiration tube connectable to said phacoemulsification probe to aspirate a cataract from an eye;
an irrigation tube; and
a compliant tube section connectable to an end of said irrigation tube and to said phacoemulsification probe, wherein said compliant tube section has a section diameter larger than a tube diameter of said irrigation tube, and is more compliant than a cornea of the eye.

10. The system according to claim 9 and wherein said compliant tube section is formed of silicone with a Shore value of 70-80.

11. The system according to claim 9 and wherein said compliant tube section has a length of 3-10 cm long.

12. The system according to claim 9 and wherein said compliant tube section is formed of a material more compliant than a material of said irrigation tube.

13. An irrigation tube for a phacoemulsification probe used for aspirating a cataract from an eye, the irrigation tube comprising:
a long tube portion; and
a compliant tube section connectable to an end of said long tube portion and to said phacoemulsification probe, wherein said compliant tube section has a section diameter larger than a tube diameter of said irrigation tube, and is more compliant than a cornea of the eye.

14. The tube according to claim 13 and wherein said compliant tube section is formed of silicone with a Shore value of 70-80.

15. The tube according to claim 13 and wherein said compliant tube section has a length of 3-10 cm long.

16. The tube according to claim 13 and wherein said compliant tube section is formed of a material more compliant than a material of said irrigation tube.
